(19) United States
(12) Patent Application Publication
(10) Pub. No.: US 2014/0030313 A1
(43) Pub. Date: Jan. 30, 2014

(54) TREATMENT OF MENSTRUAL PAIN AND EXCESSIVE MENSTRUAL BLOOD LOSS BY INTRAVAGINAL ADMINISTRATION OF A LOW DOSE OF ANTIFIBRINOLYTIC OR HEMOSTATIC AGENT IN COMBINATION WITH NON-STEROIDAL ANTI-INFLAMMATORY DRUG

(71) Applicant: ARSTAT Inc., Flemington, NJ (US)

(72) Inventor: Arkady RUBIN, Flemington, NJ (US)

(73) Assignee: ARSTAT Inc., Flemington, NJ (US)

(21) Appl. No.: 14/041,501

(22) Filed: Sep. 30, 2013

Related U.S. Application Data
(63) Continuation of application No. PCT/US2012/031227, filed on Mar. 29, 2012.
(60) Provisional application No. 61/472,904, filed on Apr. 7, 2011.

(51) Int. Cl. A61K 9/00; A61K 38/08; A61K 38/57; A61K 31/24; A61K 31/192; A61K 31/196

(52) U.S. Cl. CPC A61K 9/0036; A61K 31/192; A61K 31/196; A61K 38/57; A61K 31/24; A61K 38/08
USPC 424/432; 514/570; 514/567; 514/569; 514/561; 514/13.7; 514/533

(57) ABSTRACT

A method for relieving menstrual pain and reducing menstrual blood loss in a female is provided. The method comprises administering intravaginally to the female a first therapeutically effective amount of a non-steroidal anti-inflammatory drug (NSAID) that relieves menstrual pain and a second therapeutically effective amount of an active agent that reduces menstrual blood loss, wherein the NSAID and the active agent are simultaneously administered by the same drug delivery device, wherein the active agent is any one of an antifibrinolytic agent and a hemostatic agent.

### CROSS REFERENCE TO RELATED APPLICATIONS

This application is a Continuation of international application PCT/US2012/031227 filed on Mar. 29, 2012, which claims the benefit of US provisional patent application No. 61/472,904 filed on Apr. 7, 2011, the contents of which are incorporated by reference.

### TECHNICAL FIELD

The present invention relates to the treatment of female menstrual disorders.

More specifically, the present invention relates to the pharmacological treatment of menstrual pain accompanied with excessive menstrual blood loss. Further, the present invention relates to the pharmacological treatment of menstrual pain accompanied with excessive menstrual blood loss by simultaneous intravaginal administration of a non-steroidal anti-inflammatory drug in combination with a low dose of an antifibrinolytic or hemostatic agent.

### BACKGROUND

Menstrual disorders affect the lives of millions of women. Painful menstrual periods associated with heavy menstrual bleeding often require medical attention and initiation of appropriate therapy.

Painful menstrual periods may be clinically diagnosed as primary or secondary dysmenorrhea. Primary dysmenorrhea is defined as painful menstrual periods in women with normal pelvic anatomy. It is characterized by crampy pelvic pain beginning shortly before or at the onset of menstrual periods and lasting for one to three days. Dysmenorrhea also may be secondary to pelvic organ pathology.¹ Reported dysmenorrhea prevalence rates range from 43% to 90%. The variability in these estimates is explained by the varying methods of data collection, definitions of dysmenorrhea and populations studied.²

Heavy menstrual bleeding is defined as menorrhagia when the menstrual blood loss (MBL) exceeds 80 mL per menstrual cycle. In real-world practice, if a woman's periods are so heavy or so long that she finds them distressing, then she is experiencing heavy menstrual bleeding. One-third of all women report heavy menstrual bleeding at some point in their lives, and in Western countries about 5% of reproductive-aged women seek treatment for it annually.³

With the high prevalence of dysmenorrhea and menorrhagia, one may expect that a substantial number of women are suffering from both diseases concomitantly. Days of painful menstrual periods in conjunction with excessive menstrual blood loss may also be experienced by women not clinically diagnosed with either condition, or diagnosed with only one of them (e.g., clinically diagnosed menorrhagia in the absence of dysmenorrhea, or clinically diagnosed dysmenorrhea in the absence of menorrhagia). In North America and Europe, dysmenorrhea and menorrhagia are often treated by off-label use of approved hormonal contraceptives. For a number of women, the treatments may not be acceptable due to known contraindications, hormone-related adverse events and/or undesirable changes in the menstrual bleeding pattern, including unpredictable intra-cyclic bleeding, irregular menstrual periods and/or the development of amenorrhea.^{4,5,6} Due to known safety issues, danazol is rarely considered as a viable pharmacological treatment option. Surgical removal of the uterus (e.g., hysterectomy) may be considered for women with severe, refractory dysmenorrhea and menorrhagia. Yet, this is a radical treatment option with known undesirable consequences, including loss of fertility, surgical morbidity, as well as entailing high cost. There is limited evidence supporting minimally invasive methods of endometrial destruction as efficacious treatment options for dysmenorrhea and menorrhagia.^{1,7}

Non-steroidal anti-inflammatory drugs (NSAIDs) are considered the best-established established and the most appropriate initial therapy for dysmenorrhea.^{1,8,9,10,11,12,13,14,15,16} The following NSAIDs are commonly prescribed for the treatment of pain associated with dysmenorrhea: (1) Mefenamic acid, (Ponstel®); (2) Ibuprofen (Motrin®, Advil®); (3) Diclofenac potassium (Cataflam®); (4) Naproxen sodium (ANAPROX®/ANAPROX® DS); (5)Ketoprofen (Orudis®); (6) Meclofenamate sodium.^{17,18,19,20,21,22,23} Ibuprofen, naproxen and ketoprofen are recommended drugs.^{11,12} In the published meta-analysis, ibuprofen was singled out as the drug having the most favorable risk-benefit ratio.¹⁶

Oral NSAIDs have been shown to reduce MBL.^{6,25,26} The NSAIDs' ability to reduce MBL is related to the established relationship of endomyometrial prostaglandins to the genesis of menorrhagia. The most extensively studied NSAIDs, the fenamates, inhibit prostaglandin synthesis and bind to prostaglandin receptors which are significantly increased in women with menorrhagia²⁴.

Many patients desire greater reduction of the amount of menstrual flow than what is typically achievable using recommended doses of oral NSAIDs.²⁹ To ensure greater reduction of menstrual blood loss, the maximal NSAID doses must often be administered.⁴ This leads to undesirable side effects such as diarrhea, nausea, vomiting, stomach pain, constipation, and allergic reactions, and is not optimal, particularly if a much lower NSAID dose is sufficient to alleviate menstrual pain. As an example, a high dose of ibuprofen (800 mg every 8 hours) has been recommended for MBL reduction⁵, while a lower dose may be sufficient to alleviate menstrual pain. A recommendation in the FDA-approved class labeling for NSAIDs is to use the lowest effective dose for the shortest duration possible.²¹

In addition, NSAIDs demonstrate inferior efficacy in reducing MBL when compared to another drug widely used for treatment of menorrhagia, oral tranexamic acid. Oral tranexamic acid is marketed in the U.S. as Lysteda® and both within and outside the U.S. as Cyklokapron®. As is reported in the Lysteda label, tranexamic acid is a synthetic lysine amino acid derivative which diminishes the dissolution of hemostatic fibrin by plasmin. In the presence of tranexamic acid, the lysine receptor binding sites of plasmin for fibrin are occupied, preventing binding to fibrin monomers, thus preserving and stabilizing fibrin's matrix structure.²⁷ The antifibrinolytic activity of tranexamic acid results in inhibition of the dissolution of clots.²⁸ For many women, oral tranexamic acid is an efficacious treatment option. Clinical studies indicated that a 3900 mg/day regimen (marketed in the US as Lysteda) meets MBL reduction targets established by the FDA and significantly reduces limitations on social, leisure and physical activities.^{27,30}

However, the treatment-induced changes in MBL established in the Lysteda clinical trials may be not satisfactory for some women, and many patients may desire even greater reduction of the amount of menstrual flow. As was noted in the medical review of the Lysteda NDA, less than half (44%) of subjects returned to normal MBL after treatment (i.e., achieved a mean on-treatment MBL of less than 80 mL). There were no statistically significant differences between tranexamic acid and placebo treatment with regard to reduction of large stains, small and large clots as well as for changes in serum ferritin levels.⁶ The latter endpoint is particularly meaningful for women with impaired iron status and/or clinically-diagnosed anemia frequently associated with menorrhagia.

In the evaluation of tranexamic acid in the treatment of menorrhagia performed in 2000 by the European Agency for the Evaluation of Medicinal Products (EMEA), a dose-dependent increase in efficacy was noted. The same review recommended a daily dose of 3-4 g/day and indicated that the risk of gastrointestinal adverse events is increased at 6 g/day.³¹ While the FDA-approved Lysteda regimen (up to 3.9 g/day) is within the aforementioned recommended dosing range, certain Warnings and Precautions—dose adjustment in women with renal impairment; increase in the risk of blood clots, stroke, or myocardial infarction in the event of concomitant therapy with hormonal contraceptives; the possibility of severe allergic reactions; and visual or ocular adverse effects⁶—reflect regulatory concerns regarding Lysteda's safety. In the risk-benefit assessment, the FDA medical reviewer suggested a 50% dose reduction for women who do not tolerate the common adverse events associated with the approved treatment regimen.³⁰

A possibility of combining oral NSAID and oral tranexamic acid treatments has been suggested.⁶ It may be assumed that the currently-approved doses of 3.9 mg/day for Lysteda (US) and 3.0 mg/day for Cyklokapron® (ex-US) would be used.^{27,29} A combination oral tablet containing the standard doses of tranexamic acid (500 mg) and NSAID mefenamic acid (250 mg) is marketed in India (under Gynameno-Plus® and other trade names). These treatment modalities do not take into consideration the substantial contribution of the NSAID component to the MBL reduction. As a result, they use doses of tranexamic acid which are much greater than needed for adequate control of excessive MBL, resulting in increased incidence of adverse events.

Taken together, the clinical evidence indicates that the efficacy of oral NSAIDs in the treatment of dysmenorrhea and oral tranexamic acid in the treatment of menorrhagia must be weighed against the potentially disturbing side effects associated with these medications. When administered concomitantly via the conventional oral route at approved doses, the drugs may raise safety concerns.

In addition to the parenteral drug delivery routes (oral and intravenous), local administration of tranexamic acid has been studied. Efficacy of this route was established in a number of clinical studies.^{32,33,34} In various clinical settings, placebo-adjusted differences in blood loss (indicating a direct drug effect) ranged from 55 mL to 750 mL.^{33,34,35,36} The lower limit of the reported range is above the blood loss decrease (50 mL) considered by the FDA as a desirable menstrual blood loss reduction target.²⁷

Local administration of tranexamic acid is proven to be efficacious even with very low, sometimes undetectable, circulating drug levels. For example, use of mouthwash containing tranexamic acid was shown to reduce the incidence of postoperative bleeding complications when compared to placebo.³² Such an effect was achieved despite a small amount of tranexamic acid being administered and its short residence time in the oral cavity.⁵⁷ After administration of an oral tablet of tranexamic acid, mean drug plasma concentrations (7 mcg/mL) reached therapeutic levels, whereas the drug was not detected in saliva samples. After a mouth rinse, plasma concentrations remained below 2 mcg/mL while the saliva concentrations reached a much higher maximum level (above 200 mcg/mL) and remained at a therapeutic level for more than two hours.^{32,37} Reduction of the postoperative blood loss without detectable plasma drug levels was also reported for other locally administered antifibrinolytic drugs, specifically aprotinin.³⁴

A number of studies investigated fibrinolytic enzyme systems in menstrual blood.^{38,39,40,41,42,43,44,45} Results of one of these studies suggest that high levels of menstrual plasminogen activator and plasmin are most likely derived from the endometrium, with significantly greater levels in women with excessive bleeding when compared to those with normal menstrual volumes. Notably, no such differences could be found in the peripheral blood. Both plasminogen activator and plasmin activity in menstrual blood were significantly higher than those in peripheral blood, irrespective of the intensity of the women's menstrual bleeding.³⁹

Local delivery of NSAIDs for pain relief was also studied. For example, recent clinical programs (Pennsaid®, 1.5% w/w diclofenac sodium topical solution in treatment of knee osteoarthritis, developed by Covidien Inc.⁴⁷; Ketotransdel®, ketoprofen 10% cream, for the reduction of pain associated with acute soft tissue injuries, developed by Transdel Pharmaceuticals, Inc.⁴⁸) resulted in excellent efficacy outcomes and significant reduction of treatment-related gastrointestinal adverse events when compared to the oral route of drug administration. Notably, local delivery of NSAIDs ensured anti-inflammatory and analgesic effects despite minimal drug concentrations in blood; the estimated systemic absorption for one of the referenced compounds was about 1-2% of a comparable oral dose.⁴⁸

Feasibility of the intravaginal delivery of NSAIDs was confirmed in preclinical testing of naproxen and ketorolac in rabbits as described in U.S. Pat. No. 6,086,909. Also, data from a Phase 2 trial in women suffering from primary dysmenorrhea indicated that intravaginal delivery of mefenamic acid resulted in pain relief equivalent to that achieved with the oral capsules, despite administration at 20% of the oral dose. In addition to significant reduction of the dose of the drug required, the intravaginal mefenamic acid tampons demonstrated the potential to reduce the time until pain relief was achieved.⁴⁹

Effectiveness of intravaginal drug delivery is supported by a substantial body of evidence. It is established for estrogens used to treat vaginal atrophy and related symptoms⁵⁰, as well as osteoporosis and other menopausal symptoms.⁵¹ Other examples of compounds with greater effects after intravaginal administration include misoprostol for cervical ripening⁵², a danazol ring for the treatment of infiltrating endometriosis⁵³, and a progesterone gel.^{54,55} The contraceptive efficacy of levonorgestrel(LNG)-containing intrauterine system, Mirena® with 20 mcg/day LNG delivery, is at least comparable to that reported for the LNG-only pill delivering a 50% greater daily dose. As noted in the Mirena NDA Medical review, serum concentrations of levonorgestrel for Mirena are approximately one-tenth the serum concentration produced by an oral contraceptive containing 0.1 mg LNG and about half of that produced by the Norplant® system. The local endometrial concentrations, however, are over 100 times higher in Mirena users than in users of oral contraceptives containing 0.25 mg LNG.⁵⁶

### SUMMARY

Certain embodiments disclosed herein provide pharmaceutical compositions, delivery devices and methods for effectively relieving menstrual pain and reducing excessive menstrual blood loss (MBL) without the undesirable side effects of current oral medications, by providing simultaneous intravaginal delivery of a non-steroidal anti-inflammatory drug (NSAID) in combination with an antifibrinolytic or hemostatic agent. As disclosed herein, local administration of a combination of low doses of an NSAID and an antifibrinolytic or hemostatic agent can be rendered safe and efficacious if it utilizes intravaginal drug delivery. Drug delivery devices of the present invention allow achieving simultaneous drug delivery directly to the affected tissues that are close to the vagina (e.g., uterine cavity, uterine muscle layers) during menstrual periods. Any form of drug delivery device which will effectively deliver the treatment agents to the vaginal endothelium and adjacent tissues is intended to be included within the scope of this invention. Non-limiting examples of useful intravaginal drug delivery devices include vaginal ring, vaginal tablet, pessary, ovule, suppository, vaginal sponge, diaphragm, pad, tampon, foam, cream, ointment, and gel.

According to certain embodiments, effective local concentrations of drugs are achievable with doses much lower than those administered intravenously, or by the oral route. When used according to the method of the invention, levels of NSAID and antifibrinolytic or hemostatic agent in the systemic circulation are greatly reduced, possibly below detectable limits, leading to a lower incidence of adverse events, such as diarrhea, nausea, vomiting, stomach pain, upset stomach, constipation, heartburn, allergic reactions, disturbance of color, sharpness, or field of vision, etc. The local administration of an antifibrinolytic or a hemostatic agent (e.g., tranexamic acid) may also eliminate risk of systemic toxicity and thrombloembolism—well known risks associated with their oral and intravenous administration.

The relatively high local tissue concentrations of NSAID and antifibrinolytic or hemostatic agent achievable by the pharmaceutical compositions, delivery devices and methods of the present invention ensure a shorter time to realization of the desired pharmacodynamic effects (e.g., inhibition of prostaglandin synthesis and binding to prostaglandin receptors by NSAID), reduction of plasminogen activator and plasmin levels, and formation of strong and stable blood clots, etc. by the antifibrinolytic or hemostatic agent.

Since the menstrual pain originates from the uterine muscle layer, the local effects of NSAIDs are very important for the pain's treatment. The published findings also suggest that plasminogen activator and plasmin activity in menstrual blood are significantly higher than those in peripheral blood irrespective of the intensity of the women's menstrual bleeding.³⁹

Better compliance by women using intravaginal devices of the invention (as compared, e.g., to oral tablets) is also expected.

While the exact intravaginal doses for each drug useful in the pharmaceutical compositions, delivery devices and methods of the present invention are going to be determined in clinical trials, the possibility of a drastic dose decrease, relative to the currently approved oral doses, with no compromise (but rather improvement) in the relief of menstrual pain and in the reduction of MBL is surprising and new. With an expected decrease in drug-related adverse events, this treatment modality can be considered as the first treatment option in the management of menstrual pain accompanied with excessive MBL. Also surprising and new is the possibility of achieving a therapeutic effect (manifested in the relief of menstrual pain and in the reduction of MBL) in the absence of detectable plasma concentration levels, or in the presence of circulating levels of the drugs much lower than those reported after administration of oral tablets.

While the exact intravaginal doses for each drug useful in the pharmaceutical compositions, delivery devices and methods of the present invention are going to be determined in clinical trials, initial dose selection is driven by a number of factors, including, but not limited to, the potency of the specific compounds involved, the volume of menstrual flow and the presence of clots, stains, and the severity of the symptoms associated with menstruation, as well as specific patient characteristics (age, weight, presence of anemia, duration of the disease, etc.). The dose selection also takes into account the effect of a given NSAID in reducing the volume of MBL. For NSAIDs which cause reduction in the volume of MBL, the dose of co-administered antifibrinolytic or hemostatic agent can be lowered. Relative severity of menstrual pain and the volume of MBL in a given patient must also be taken into account. For example, in women with severe dysmenorrhea and relatively modest MBL, addition of a very small dose of antifibrinolytic or hemostatic agent to the potent dose of NSAID should be considered. If, however, menstrual pain is less than severe and MBL is high, then a relatively small dose of NSAID is combined with a substantial complementary dose of antifibrinolytic or hemostatic agent. In all cases, the goal is an effective treatment of both conditions with an appropriate dose of each component. Following the intravaginal administration of a combination of an NSAID and an antifibrinolytic or hemostatic agent according to the present invention, reduction of MBL is expected to exceed clinical targets established by the FDA³⁰ in the treatment of menorrhagia.

Non-limiting examples of NSAIDs useful according to the present invention include ibuprofen, naproxen, diclofenac, ketoprofen, mefenamic acid, meclofenamate sodium, and metabolites thereof.

Non-limiting examples of useful antifibrinolytic and hemostatic agents according to the present invention include tranexamic acid, ε-amino-caproic acid, aprotinin, antipan, gabexate mesilate, pepstatin, leupeptin, chymostatin, and metabolites thereof.

The optimal treatment duration will also be determined during the clinical trials. In one embodiment of the invention, the drug administration starts at the onset of the menstrual period and last for several days, until the end of menstrual period or at least until the end of painful and/or heavy menstrual bleeding.

### DETAILED DESCRIPTION

The embodiments disclosed herein are only examples of the many possible advantageous uses and implementations of the innovative teachings presented herein. In general, statements made in the specification of the present application do not necessarily limit any of the various claimed embodiments. Moreover, some statements may apply to some inventive features but not to others.

### Definitions

A vaginal ring (also known as an intravaginal ring) is a polymeric drug delivery device providing controlled release of drug(s) to the vagina over an extended period of time.

Menstrual flow is defined as encompassing menstrual blood and/or menstrual fluid.

Menstrual pain relief is defined as a decrease in the severity of menstrual pain when compared to the pre-treatment conditions. Menstrual pain relief may be complete (when a woman does not experience any pain) or partial (when a woman experiences less severe pain).

A therapeutically effective amount of NSAID is defined as the amount of the drug that results in significant (at least, 20%) changes in a 4-point verbal rating scale ranging from 0 (no pain) to 3 (severe pain) and/or a 100-mm visual analog scale of severity of the menstrual pain (see reference 58) when compared to the pre-treatment conditions. A therapeutically effective amount of an antifibrinolytic or hemostatic agent is defined as the amount of the drug that results in significant (at least, 15%) change in the volume of menstrual blood loss when compared to the pre-treatment conditions. A daily dose of an NSAID or an antifibrinolytic or hemostatic agent is defined as amount of the drug released from the intravaginal device or composition on a daily basis.

Certain disclosed embodiments provide a method for relieving menstrual pain and reducing menstrual blood loss in a female comprising simultaneously administering intravaginally to the female a first therapeutically effective amount of a non-steroidal anti-inflammatory drug (NSAID) and a second therapeutically effective amount of an antifibrinolytic or hemostatic agent. In one embodiment, the NSAID and the antifibrinolytic or hemostatic agent are administered in the same composition. In one embodiment, the NSAID and the antifibrinolytic or hemostatic agent are delivered simultaneously on a delivery device providing simultaneous drug release to local affected tissues.

In one embodiment, the disclosed method can be useful for relieving menstrual pain and reducing menstrual blood loss in females who have menstrual bleeding of less than 80 ml per menstrual cycle as well as in females who have menstrual bleeding of more than 80 ml per menstrual cycle.

The method disclosed herein can be useful for relieving menstrual pain and reducing menstrual blood loss in females who have various conditions, including, without limitation, primary dysmenorrhea, secondary dysmenorrhea, menorrhagia, idiopathic menorrhagia, cyclic heavy menstrual bleeding, dysfunctional uterine bleeding, and anemia. In one embodiment, the NSAID and the antifibrinolytic or hemostatic agent are administered from the onset of menstrual bleeding until the resolution of related symptoms or the end of the menstrual period.

In one embodiment, upon administration of the NSAID and the antifibrinolytic or hemostatic agent, the amount of the NSAID and the antifibrinolytic or hemostatic agent in the female's systemic circulation is below detection levels. In conjunction with the above methods, certain embodiments also include medicated intravaginal devices comprising a first therapeutically effective amount of a non-steroidal anti-inflammatory drug (NSAID) and a second therapeutically effective amount of an antifibrinolytic or hemostatic agent.

Non-limiting examples of the intravaginal delivery devices useful according to the present invention include vaginal ring, vaginal tablet, pessary, ovule, suppository, vaginal sponge, diaphragm, pad, tampon, foam, cream, ointment, and gel. In one embodiment, the intravaginal delivery device is a vaginal ring.

In one embodiment, the NSAID and the antifibrinolytic or hemostatic agent can be mixed throughout the vaginal ring. The NSAID and the antifibrinolytic or hemostatic agent can be, e.g., distributed uniformly throughout the vaginal ring, or encapsulated in a part of the vaginal ring, or located at the center of the vaginal ring, or membranes of the NSAID and the antifibrinolytic or hemostatic agent can be placed between an unmedicated core and a metering layer of the vaginal ring.

In an embodiment, a pharmaceutical composition for intravaginal administration is provided. The disclosed composition includes a first therapeutically effective amount of a non-steroidal anti-inflammatory drug (NSAID) and a second therapeutically effective amount of an antifibrinolytic or hemostatic agent. Non-limiting examples of useful pharmaceutical compositions include liquid, tablet, foam, cream, ointment, and gel.

In one embodiment, the NSAID is able to reduce the volume of menstrual blood loss. Non-limiting examples of NSAIDs useful according to the present invention include ibuprofen, naproxen, diclofenac, ketoprofen, mefenamic acid, meclofenamate sodium, and metabolites thereof. In one embodiment, the daily NSAID dose ranges from 1 mg to 500 mg. In one embodiment, the NSAID is ibuprofen and the daily dose of ibuprofen ranges from 10 mg to 400 mg. In one embodiment, the NSAID is naproxen and the daily dose of naproxen ranges from 10 mg to 300 mg. In one embodiment, the

NSAID is diclofenac and the daily dose of diclofenac ranges from 5 mg to 25 mg. In one embodiment, the NSAID is ketoprofen and the daily dose of ketoprofen ranges from 5 mg to 25 mg. In one embodiment, the NSAID is mefenamic acid and the daily dose of mefenamic acid ranges from 10 mg to 125 mg.

Non-limiting examples of antifibrinolytic or hemostatic agents useful according to the present invention include tranexamic acid, ε-amino-caproic acid, aprotinin, antipan, gabexate mesilate, pepstatin, leupeptin, chymostatin, and metabolites thereof. In one embodiment, the daily dose of antifibrinolytic or hemostatic agent does not exceed 1 g. In another embodiment, the daily dose of antifibrinolytic or hemostatic agent ranges from 50 mcg to 500 mg. In one embodiment the antifibrinolytic agent is tranexamic acid and the daily dose of tranexamic acid ranges from 100 mg to 250 mg. In one embodiment the antifibrinolytic agent is ε-amino-caproic acid and the daily dose of ε-amino-caproic acid ranges from 250 mg to 400 mg. In one embodiment the antifibrinolytic agent is aprotinin and the daily dose of aprotinin ranges from 150 mg to 300 mg.

The disclosed embodiments are further described and demonstrated by way of the following non-limiting examples. However, the use of these and other examples is illustrative only and in no way limits the scope and meaning of the invention or of any exemplified term. Likewise, the invention is not limited to any particular preferred embodiments described herein. Indeed, many modifications and variations of the invention may be apparent to those skilled in the art upon reading this specification, and such variations can be made without departing from the invention in spirit or in scope. The invention and the various embodiments are therefore to be limited only by the terms of the appended claims along with the full scope of equivalents to which those claims are entitled.

### Example 1

The vaginal ring serving as a drug delivery device comprises a supporting ring free of active drugs. The next (second) layer contains medications selected for treatment of painful menstrual periods (NSAID) accompanied with excessive menstrual blood loss (tranexamic acid or another antifibrinolytic or hemostatic agent). This layer is coated with the third, drug-free layer. A detailed description of such vaginal ring and suitable manufacturing methods can be found in U.S. Pat. No. 4,822,616.

Per U.S. Pat. No. 4,822,616, the supporting ring is made from a physiologically acceptable synthetic resin, such as, e.g., polyethylene, RTV silicone elastomers, LTV silicone elastomers, polyamides and polytetrafluoroethylene. The second layer with active medications comprises a pharmaceutically acceptable resin from which the drugs are released. A preferred embodiment consists of the combination of drugs and LTV silicone elastomer with a composition also described in the patent. Any LTV silicone elastomer is used in the third layer. The proposed vaginal ring ensures release of the active drugs within the limits of the dosage required for the desired relief of menstrual pain and reduction of menstrual blood loss.

In one embodiment, the second layer is medicated with diclofenac in an amount adequate to release the drug in a rate of or 5-25 mg/day and with tranexamic acid in an amount adequate to release the drug in a rate of 100-250 mg/day. In another embodiment, the second layer is medicated with diclofenac in an amount adequate to release the drug in a rate of or 5-25 mg/day and with ε-amino-caproic acid in an amount adequate to release the drug in a rate of 250-400 mg/day. In yet another embodiment, the second layer is medicated with diclofenac in an amount adequate to release the drug in a rate of or 5-25 mg/day and with aprotinin acid in an amount adequate to release the drug in a rate of 150-300 mg/day.

### Example 2

The vaginal ring serving as a drug delivery device comprises active drugs selected for treatment of painful menstrual period (NSAID) accompanied with excessive menstrual blood loss (tranexamic acid or another antifibrinolytic or hemostatic agent) and a delivery module. The delivery module comprises (a) a reservoir for storing the active drugs, (b) a rate controller or wall that is formed of styrene-butadiene copolymer that maintains the prescribed rate of drug release throughout the life of system, (c) an energy source or the concentration of active drugs in the reservoir that provides the driving means for transferring the active drugs from a higher amount in the reservoir to the rate controller, (d) an inner mass transfer conductor for housing the active drugs in reservoir, and (e) a portal that provides the exit from the drug delivery module to the tissues. Detailed description of such vaginal ring and its manufacturing process can be found, for example, in U.S. Pat. No 4,250,611.

In one embodiment, the delivery module of the vaginal ring contains ketoprofen in an amount adequate to release the drug in a rate of 5-25 mg/day and tranexamic acid in an amount supporting the drug release at a rate of 100-250 mg/day. In another embodiment, the delivery module of the vaginal ring contains ketoprofen in an amount adequate to release the drug in a rate of 5-25 mg/day and ε-amino-caproic acid in an amount supporting the drug release at a rate of 250-400 mg/day. In yet another embodiment, the delivery module of the vaginal ring contains ketoprofen in an amount adequate to release the drug in a rate of 5-25 mg/day and aprotinin in an amount supporting the drug release at a rate of 150-300 mg/day.

### Example 3

The vaginal ring serving as a drug delivery device is a ring-shaped solid carrier made of silicone rubber (polysiloxane) or other suitable material. The ring has a homogenous design with active drugs dispersed in the carrier. Detailed description of such vaginal ring can be found, for example, in U.S. Pat. No 5,869,081.

Per U.S. Pat. No 5,869,081, the vaginal ring provides sustained release of the medications and results in low circulatory levels of the drugs, while concentrating its biological effect on a regional level.

In one embodiment, the carrier contains ibuprofen in an amount adequate to release the drug in a rate of or 100-200 mg/day and tranexamic acid in an amount supporting the drug release at a rate of 100-250 mg/day. In another embodiment, the carrier contains ibuprofen in an amount adequate to release the drug in a rate of or 100-200 mg/day and ε-amino-caproic acid in an amount supporting the drug release at a rate of 250-400 mg/day. In yet another embodiment, the carrier contains ibuprofen in an amount adequate to release the drug in a rate of or 100-200 mg/day and aprotinin in an amount supporting the drug release at a rate of 150-300 mg/day.

### Example 4

The vaginal ring serving as a drug delivery device is a ring-shaped solid carrier designed for the simultaneous release of at least two active substances. The ring has at least two reservoirs each containing a different agent. The reservoirs are substantially tubular and at least one end of such reservoir is attached to the end of another reservoir by means of a plug that does not permit transport of the agents by diffusion or by any other method. The reservoirs are assembled together to form a drug release system. Detailed description of such vaginal ring can be found, for example, in U.S. Pat. No 4,596,576.

Per U.S. Pat. No. 4,596,576, the vaginal ring provides sustained release of the two agents in a fixed ratio over a lengthy period.

In one embodiment, the carrier contains ibuprofen in an amount adequate to release the drug in a rate of 100-200 mg/day and tranexamic acid in an amount supporting the drug release at a rate of 100-250 mg/day. In another embodiment, the carrier contains ibuprofen in an amount adequate to release the drug in a rate of 100-200 mg/day and ε-amino-caproic acid in an amount supporting the drug release at a rate of 250-400 mg/day. In yet another embodiment, the carrier contains ibuprofen in an amount adequate to release the drug in a rate of 100-200 mg/day and aprotinin in an amount supporting the drug release at a rate of 150-300 mg/day.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

All patents, applications, publications, test methods, literature, and other materials cited herein are hereby incorporated by reference in their entirety as if physically present in this specification.

### REFERENCES

¹French Linda, M. D. American Family Physician, 2005 Jan 15; 71(2): 285-291.
²MerckMedicus Modules: Dysmenorrhea—Epidemiology; accessed on Jul. 25, 2010 at http://www.merckmedicus.com/pp/us/hcp/diseasemodules/dysmenorrhea/epidemiology. jsp (reference on file)
³Heavy menstrual bleeding: Assessing Impact, Evaluating Management Options; Supplement to OBG Management, October 2009; accessed at http://www.obgmanagement.com/PDF/supp/OBG heavybleeding.pdf (reference on file)
⁴David Chelmow Non-surgical Options for Menorrhagia. OBG Management November 2005 (Vol. 17, No.11)
⁵Kaunitz A M. Modern Management of Heavy Menstrual Bleeding (presentation on file)
⁶National Collaborating Centre for Women's and Children's Health. Heavy menstrual bleeding. Royal College of Obstetricians and Gynaecologists (RCOG); 2007 Jan.

⁷Barbara S, Apgar et al. Treatment of Menorrhagia. American Family Physician—Volume 75, Issue 12 (June 2007)
⁸Coco AS. MD. Primary dysmenorrhea. American Family Physician 1999; 60: 489-496.
⁹Proctor M, Farquhar C. Dysmenorrhoea. In: Clinical evidence. Issue 11. London: BMJ Publishing, 2004.
¹⁰Ronald T. Burkman, MD; Sandra A. Carson, MD. Noncontraceptive Health Benefits of Progestin-Only Contraceptive Agents. The Female Patient |Vol 33 August 2008
¹¹Dawood, Y, Glob. libr. women's med., (ISSN: 1756-2228) 2008; DOI 10.3843/GLOWM.10009a, Dysmenorrhea The Global Library of Women's Medicine, 2008; accessed at http://www.glowm.com/index.html?p=glowm.cml/section_view&articleid=9 (reference on file)
¹²RECOMMENDATIONS FOR THE TREATMENT OF DYSMENORRHEA. 2000. University of Texas at Austin School of Nursing, Family Nurse Practitioner Program.
¹³ACOG Practice Bulletin No. 51. Chronic pelvic pain. Obstet Gynecol. 2004 Mar;103(3):589-605.
¹⁴Primary Dysmenorrhea Consensus Guideline Society of Obstetricians and Gynaecologists of Canada, No 169, December 2005.
¹⁵Marjoribanks J, Proctor M L, Farquhar C. Nonsteroidal anti-inflammatory drugs for primary dysmenorrhoea. In: The Cochrane Library, Issue 2,2006.
¹⁶Zhang W Y, Li Wan Po A. Efficacy of minor analgesics in primary dysmenorrhoea: a systematic review. Br J Obstet Gynaecol 1998;105:780-789.
¹⁷Ponstel®. Full Prescribing Information.
¹⁸Motrin®. Full Prescribing Information.
¹⁹Ibuprofen Oral: Dosage, Uses and Warnings (reference on file).
²⁰Cataflam® Full Prescribing Information.
²¹Naproxen Sodium. Full Prescribing Information.
²²Ketoprofen Full Prescribing Information.
²³Meclofenamate sodium. Full Prescribing Information.
²⁴Oeller M K, Rees M C P. Menorrhagia: an update. Acta Obstet Gynecol Scand 2003; 82: 405-422
²⁵Duckitt K., Collins S. Menorrhqagia. Clinical Evidence BMJ publishing group 2008.
²⁶Lethaby A, et al. Cochrane Database Syst Rev. 2007;(4):CD000400.
²⁷Lysteda®. Full Prescribing Information.
²⁸Joseph Y. Lee, et al. Treatment of Menorrhagia with Tranexamic Acid. J Soc Obstet Gynaecol Can 2000;22(10):794-8
²⁹Cyklokapron° . Summary of Product Characteristics
³⁰ Center for Drug Evaluation and Research. Application Number 22-430. Lysteda® NDA Medical review; accessed at http://www.accessdata.fda.qov/druqsatfda docs/nda/2009/022430s000medr.pdf (reference on file)
³¹Overall Summary of the Scientific Evaluation of Cycle-f, EMEA, 2000.
³²Dunn C J, Goa K L. Tranexamic acid: a review of its use in surgery and other indications. Drugs. 1999;57:1005-32.
³³Jabalameli M., Zakeri K. Evaluation of Topical Tranexamic Acid on Intraoperative Bleeding in Endoscopic Sinus Surgery Iran J Med Sci December 2006; Vol 31 No 4 221
³⁴De Bonnis M. et al. Topical use of tranexamic acid in coronary artery bypass operations: a double-blind, prospective, randomized, placebo-controlled study. J Thorac Cardiovasc Surg. 2000 Mar;119(3):575-80
³⁵Fawzy H, et al. Can local application of Tranexamic acid reduce post-coronary bypass surgery blood loss? A randomized controlled trial. J Cardiothorac Surg. 2009 Jun 18;4:25
³⁶Vlessides M. Topical Tranexamic Acid Cuts Bleeding After Knee Surgery. Clinical Anesteology. February 2010|VOLUME: 36:2
³⁷Sindet-Pedersen S. Distribution of tranexamic acid to plasma and saliva after oral administration and mouth rinsing: a pharmacokinetic study. J Clin Pharmacol 1987;27:1005-8.
³⁸Gleeson N C et al. The effect of tranexamic acid on measured menstrual loss and endometrial fibrinolytic enzymes in dysfunctional uterine bleeding. Acta Obstetricia et Gynecologica Scandinavica 1994 73:3, 274-277
³⁹Dockeray C J. et al. The fibrinolytic enzyme system in normal menstruation and excessive uterine bleeding and the effect of tranexamic acid. Eur J Obstet Gynecol Reprod Biol. 1987 Apr;24(4):309-18
⁴⁰Rybo G. Plasminogen activators in endometrium. Acta Obstet Gynecol Scand. 1966; 45:411-449
⁴¹Hefnawi AS. et al. Fibrinolytic activity of menstrual blood in normal and menorrhagic women and in women wearing the Lippes Loop and Cu-T (200). Int J Gynaecol Obstet 1979; 16:400-407.
⁴²Hahn L. et al. Blood coagulation, fibrinolysis and plasma protein in women with normal and with excessive menstrual blood loss. Br J Obstet Gynaecol 1976; 83: 974-980.
⁴³Cole S K. Clarkson AR. Menstrual blood loss and fibrin degradation products. Br Med J 1972; 1:78-79.
⁴⁴Rees M C P et al. Coagulation factors and fibrinolytic proteins in menstrual fluid collected from normal and menorrhagic women. Br J Obstet Gynaecol 1985; 92: 1164-1168
⁴⁵Basu H K. Fibrin degradation products in sera of women with normal menstruation and menorrhagia. Br Med J 1970; 1:74-75.
⁴⁶lnternational Women's Health Update. Antifibrinolytics; Monash University
⁴⁷Analysis Shows Topical NSAID Has Potentially Better Gastrointestinal Safety Profile Than Oral NSAIDs in Treatment of Knee Osteoarthritis; accessed at http://www.businesswire.com/news/home/20101108006053/en/Analysis-Shows-Topical-NSAID-Potentially-Gastrointestinal-Safety (reference on file)
⁴⁸Transdel Pharmaceuticals presents Ketotransdel Phase 3 data . . . ; accessed at http://www.drugs.com/clinical_trials/transdel-pharmaceuticals-presents-phase-3-study-data-ketotransdel-world-pain-congress-montreal-10035.html (reference on file)
⁴⁹Medicated tampon reduces chronic menstrual pain, Medical Textiles, June. 2004, accessed at http://findarticles.com/p/articles/mi_hb5987/is_{—}2004_June/ai_n32028248/(reference on file)
⁵⁰Kingsberg S A., et al. Treating dyspareunia caused by vaginal atrophy: a review of treatment options using vaginal estrogen therapy. International Journal of Women's Health. August 2009, Volume 2009:1 Pages 105-111
⁵¹Ballagh S A. Vaginal Ring Hormone Delivery Systems in Contraception and Menopause. Clinical obstetrics and gynecology/volume 44/number 1/March 2001
⁵²Iyer V., et al. Vaginal drug delivery. ExpressPharma, 1-15 July 2008
⁵³lgarashi M. Novel vaginal danazol ring therapy for pelvic endometriosis, in particular deeply infiltrating endometriosis. Hum Reprod. 1998 Jul;13(7):1952-6.
⁵⁴Ficicioglu C. et al. High local endometrial effect of vaginal progesterone gel. Gynecological Endocrinology, Volume 18, Issue 5 May 2004, pages 240-243.
⁵⁵Kuhl H. Pharmacology of estrogens and progestogens: influence of different routes of administration. Climacteric 2005; 8(Suppl 1)3-63.
⁵⁶Center for Drug Evaluation and Research. Application Number 21-225. Mirena® NDA Medical review; accessed at Mirena® NDA Medical review; accessed at http://www.accessdata.fda.qov/druqsatfda docs/nda/2000/21-225.pdf Mirena Medr.pdf (reference on file)
⁵⁷Randall C. U. K. National Health Service. Surgical management of the primary care dental patient on warfarin. March 2007. Accessed at http://www.dundee.ac.uk/tuith/Static/info/warfarin.pdf (reference on file)
⁵⁸Caraceni A. et al., Pain Measurement Tools and Methods in Clinical Research. Journal of Pain and Symptom Management, Vol. 23 No. 3 March 2002

What is claimed is:

1. A method for relieving menstrual pain and reducing menstrual blood loss in a female comprising:
administering intravaginally to the female a first therapeutically effective amount of a non-steroidal anti-inflammatory drug (NSAID) that relieves menstrual pain and a second therapeutically effective amount of an active agent that reduces menstrual blood loss, wherein the NSAID and the active agent are simultaneously administered by the same drug delivery device, wherein the active agent is any one of an antifibrinolytic agent and a hemostatic agent.

2. The method of claim 1, wherein the drug delivery device is any one of: a vaginal ring, a vaginal tablet, a pessary, an ovule, a suppository, a vaginal sponge, a diaphragm, a pad, a tampon, foam, cream, ointment, and gel.

3. The method of claim 2, wherein the active agent and the NSAID are contained in the vaginal ring as any one of the following forms: mixed throughout the vaginal ring, distributed uniformly throughout the vaginal ring; encapsulated in a part of the vaginal ring, located at the center of the vaginal ring, and placed between an un-medicated core and a metering layer of the vaginal ring.

4. The method of claim 1, wherein the NSAID comprises any one of: ibuprofen, naproxen, diclofenac, ketoprofen, mefenamic acid, meclofenamate sodium, and metabolites thereof.

5. The method of claim 4, wherein the daily dose of the NSAID ranges from 1 mg to 500 mg.

6. The method of claim 5, wherein the daily dose of the NSAID is any one of: from 10 mg to 400 mg of the ibuprofen; from 10 mg to 300 mg of the naproxen; from 5 mg to 25 mg of the diclofenac; from 5 mg to 25 mg of the ketoprofen, and from 10 mg to 125 mg of the mefenamic acid.

7. The method of claim 1, wherein each of the antifibrinolytic agent and the hemostatic agent comprises any one of: tranexamic acid, ε-amino-caproic acid, aprotinin, antipan, gabexate mesilate, pepstatin, leupeptin, chymostatin, and metabolites thereof.

8. The method of claim 1, wherein each of the antifibrinolytic agent and the hemostatic agent of the active agent is delivered at a daily agent dose that does not exceed 1 g.

9. The method of claim 7, wherein each of the antifibrinolytic agent and the hemostatic agent is delivered at a daily agent dose ranging from 50 mcg to 500 mg.

10. The method of claim 9, wherein the daily dose of the active agent is any one of:
from 100 mg to 250 mg of the tranexamic acid, from 250 mg to 400 mg of the c-amino-caproic acid, and from150 mg to 300 mg of the aprotinin.

11. The method of claim 1, wherein the female has a condition that comprises any one of: primary dysmenorrhea, secondary dysmenorrhea, menorrhagia, idiopathic menorrhagia, cyclic heavy menstrual bleeding, dysfunctional uterine bleeding, and anemia.

12. An intravaginal drug delivery device, comprising:
a first therapeutically effective amount of a non-steroidal anti-inflammatory drug (NSAID) that relieves menstrual pain and a second therapeutically effective amount of an active agent that reduces menstrual blood loss, wherein the delivery device simultaneously delivers a daily dose of the NSAID ranging from 1 mg to 500 mg and a daily dose of the active agent not exceeding 1 g, wherein the active agent is any one of an antifibrinolytic agent and a hemostatic agent.

13. The intravaginal drug delivery device of claim 12, wherein the intravaginal drug delivery device is any one of: a vaginal ring, a vaginal tablet, a pessary, an ovule, a suppository, a vaginal sponge, a diaphragm, a pad, a tampon, foam, cream, ointment, and gel.

14. The intravaginal drug delivery device of claim 12, wherein the active agent and the NSAID are contained in the vaginal ring as any one of the following forms: mixed throughout the vaginal ring, distributed uniformly throughout the vaginal ring; encapsulated in a part of the vaginal ring, located at the center of the vaginal ring, and placed between an un-medicated core and a metering layer of the vaginal ring.

15. The intravaginal drug delivery device of claim 13, wherein the NSAID comprises any one of: ibuprofen, naproxen, diclofenac, ketoprofen, mefenamic acid, meclofenamate sodium, and metabolites thereof.

16. The intravaginal drug delivery device of claim 13, wherein each of the antifibrinolytic agent and the hemostatic agent comprises any one of: tranexamic acid, c-amino-caproic acid, aprotinin, antipan, gabexate mesilate, pepstatin, leupeptin, chymostatin, and metabolites thereof.

17. The intravaginal drug delivery device of claim 15, wherein the vaginal ring delivers a daily dose comprising any one of: from 10 mg to 400 mg of the ibuprofen, from 10 mg to 300 mg of the naproxen, from 5 mg to 25 mg of the diclofenac, from 5 mg to 25 mg of the ketoprofen, and from 10 mg to 125 mg of the mefenamic acid.

18. The intravaginal drug delivery device of claim 16, wherein the vaginal ring delivers a daily dose comprising any one of: from 100 mg to 250 mg of tranexamic acid; from 250 mg to 400 mg of the c-amino-caproic acid, and from 150 mg to 300 mg of the aprotinin.

19. A pharmaceutical composition for intravaginal administration, comprising:
a first therapeutically effective amount of a non-steroidal anti-inflammatory drug (NSAID) that relieves menstrual pain and a second therapeutically effective amount of an active agent that reduces menstrual blood loss, wherein the active agent is any one of an antifibrinolytic agent and a hemostatic agent.

20. The composition of claim 19, wherein the composition is formulated as any one of: an aqueous and a pharmaceutically acceptable solution of the active agents, wherein the formulation is in a form comprising any one of: liquid, foam, cream, ointment, and gel.

21. The composition of claim 17, wherein the composition is formulated as a tablet.

22. The composition of claim 19, wherein the NSAID comprises any of: ibuprofen, naproxen, diclofenac, ketoprofen, mefenamic acid, meclofenamate sodium, and metabolites thereof.

23. The composition of claim 19, wherein each of the antifibrinolytic and the hemostatic agent of the active agent comprises any of: tranexamic acid, c-amino-caproic acid, aprotinin, antipan, gabexate mesilate, pepstatin, leupeptin, chymostatin, and metabolites thereof.
